(19) United States
(12) Patent Application Publication
(10) Pub. No.: US 2026/0224223 A1
(43) Pub. Date: Aug. 6, 2026

(54) EMBOLIC COIL WITH GRADIENT STIFFNESS

(71) Applicant: BOSTON SCIENTIFIC SCIMED, INC., MAPLE GROVE, MN (US)

(72) Inventors: Ajay Gupta, Minneapolis, MN (US); Benjamin Wai-Man Chan, Edina, MN (US); Robert M. Wold, Maple Lake, MN (US); Grigory Severyukhin, Zimmerman, MN (US)

(73) Assignee: BOSTON SCIENTIFIC SCIMED, INC., MAPLE GROVE, MN (US)

(21) Appl. No.: 19/462,163

(22) Filed: Jan. 28, 2026

Related U.S. Application Data
(60) Provisional application No. 63/751,115, filed on Jan. 29, 2025.

(51) Int. Cl. A61B 17/12; A61B 17/00

(52) U.S. Cl. CPC A61B 17/12145; A61B 17/12109; A61B 17/1215; A61B 2017/00526; A61B 2017/00831; A61B 2017/1205

(57) ABSTRACT

An embolic coil is formed from a continuous wire and has a primary shape formed by winding the wire on a tapered mandrel such that the primary shape of the coil is a spiral having an outer diameter that increases from a first outer diameter at the proximal end to a second outer diameter at the distal end that is larger than the first outer diameter. The coil has the primary shape when constrained within a delivery catheter. The coil has a gradient stiffness profile transitioning from a first stiffness at the proximal end to a second stiffness at the distal end that is less than the first stiffness. The coil has a secondary shape after exiting the delivery catheter that includes at least one loop or fold formed from the spiral of the primary shape.

### CROSS-REFERENCE TO RELATED APPLICATIONS

This application claims the benefit of priority of U.S. Provisional Application No. 63/751,115 filed Jan. 29, 2025, the entire disclosure of which is hereby incorporated by reference.

### TECHNICAL FIELD

The disclosure pertains to medical devices and more particularly to embolic coils, and methods for using such medical devices.

### BACKGROUND

A wide variety of medical devices have been developed for medical use including, for example, medical devices utilized to create therapeutic vascular occlusions (i.e., embolizations) to prevent or treat pathological conditions in situ. These medical devices may be used in a variety of vessels and are manufactured and used according to any one of a variety of different methods. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example embolic coil includes a coil having a proximal end and a distal end and formed from a continuous wire, the coil having a primary shape formed by winding the continuous wire on a tapered mandrel such that the primary shape of the coil is a spiral having an outer diameter that increases from a first outer diameter at the proximal end to a second outer diameter at the distal end that is larger than the first outer diameter, the coil having the primary shape when constrained within a delivery catheter, wherein the coil has a gradient stiffness profile transitioning from a first stiffness at the proximal end to a second stiffness at the distal end that is less than the first stiffness, and wherein the coil has a secondary shape after exiting the delivery catheter, the secondary shape including at least one loop or fold formed from the spiral of the primary shape.

Alternatively or additionally to the embodiment above, the outer diameter in the primary shape increases uniformly from the proximal end to the distal end.

Alternatively or additionally to any of the embodiments above, the embolic coil further includes a plurality of fibers attached between windings of the spiral in the primary shape.

Alternatively or additionally to any of the embodiments above, the plurality of fibers is attached only to a proximal section of the coil.

Alternatively or additionally to any of the embodiments above, the plurality of fibers is formed from at least one of polyester, nylon, and polypropylene.

Alternatively or additionally to any of the embodiments above, the continuous wire comprises a platinum-containing alloy.

Alternatively or additionally to any of the embodiments above, the first outer diameter is between 0.02 inch and 0.025 inch and the second outer diameter is between 0.023 inch and 0.03 inch.

Alternatively or additionally to any of the embodiments above, the tapered mandrel has an oval cross-section.

An example embolization system includes a delivery catheter, an embolic coil releasably coupled to the delivery catheter, the embolic coil configured to be disposed within a lumen of the delivery catheter in a primary shape, and to form a secondary shape upon exiting the delivery catheter, the embolic coil having a proximal end and a distal end and formed from a continuous wire, the primary shape of the embolic coil formed by winding the continuous wire on a tapered mandrel such that the primary shape of the embolic coil is a spiral having an outer diameter that increases from a first outer diameter at the proximal end to a second outer diameter at the distal end that is larger than the first outer diameter, and wherein the embolic coil has a gradient stiffness profile transitioning from a first stiffness at the proximal end to a second stiffness at the distal end that is less than the first stiffness.

Alternatively or additionally to the embodiment above, the secondary shape is formed by releasing the embolic coil from the tapered mandrel and moving the embolic coil into at least one loop or fold and heat treating the secondary shape.

Alternatively or additionally to any of the embodiments above, the outer diameter in the primary shape increases uniformly from the proximal end to the distal end.

Alternatively or additionally to any of the embodiments above, the embolic coil includes a plurality of polymeric fibers attached between windings of the spiral in the primary shape.

Alternatively or additionally to any of the embodiments above, the plurality of polymeric fibers is attached only to a proximal section of the embolic coil.

Alternatively or additionally to any of the embodiments above, the continuous wire comprises a platinum-containing alloy.

Alternatively or additionally to any of the embodiments above, the first outer diameter is between 0.014 inch and 0.017 inch and the second outer diameter is between 0.016 inch and 0.019 inch.

Alternatively or additionally to any of the embodiments above, the tapered mandrel has an oval cross-section.

An example method of manufacturing an embolic coil includes providing a mandrel having a tapered outer diameter, wrapping a continuous wire around the mandrel to form a primary coil having a proximal end having a first outer diameter and a distal end having a second outer diameter larger than the first outer diameter, wherein the tapered outer diameter of the mandrel creates a gradient stiffness profile in the primary coil when released from the mandrel, removing the primary coil from the mandrel and forming a secondary shape including at least one loop or fold in the primary coil, heat treating the primary coil to set the secondary shape, and attaching a plurality of polymeric fibers between windings of the primary coil.

Alternatively or additionally to the embodiment above, the tapered outer diameter of the mandrel increases uniformly from the proximal end to the distal end.

Alternatively or additionally to any of the embodiments above, the mandrel has an oval cross-sectional shape.

Alternatively or additionally to any of the embodiments above, the first outer diameter is between 0.02 inch and 0.025 inch and the second outer diameter is between 0.023 inch and 0.03 inch.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example embolic coil wound around a tapered mandrel;

FIG. 2A illustrates a side view of the embolic coil of FIG. 1 released from the mandrel and with fibers attached;

FIG. 2B is a cross-sectional view taken through line 2B-2B in FIG. 2A;

FIG. 2C is a cross-sectional view of another embolic coil with fibers attached;

FIG. 3 illustrates the embolic coil of FIG. 2A within a delivery catheter; and

FIGS. 4A-4D illustrate example embolic coils with fibers attached in various secondary shapes.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “ and/or ” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Embolic coils are medical devices used in endovascular procedures to treat various vascular conditions. Embolic coils can be used for purposes including, for example, to close blood vessels and/or fill aneurysmal sacs. These coils may include wires formed into a primary shape and made from biocompatible materials such as platinum, platinum-iridium, or platinum-stainless steel alloys. The primary coil structure may be enhanced with secondary shaping to achieve specific therapeutic outcomes. During deployment, embolic coils may be delivered in their primary shape through a delivery catheter to a target vessel, where they may move into a secondary shape and conform to the vessel architecture. The characteristics of these coils, including their outer diameter, flexibility, and structural properties, may play a role in their performance during navigation through tortuous vasculature and their final deployment configuration.

Embolic coils are often releasably attached to a delivery system, generally a wire, for delivery of the coil to the desired site. Upon completion of satisfactory positioning, which may include partial or complete withdrawal back into the delivery catheter, the embolic coil may be detached from the delivery system and remain in the body after the delivery system is withdrawn. Another type of embolic coil is a pushable coil that is not attached to the delivery system but rather the embolic coil is simply pushed by a wire-like device through the delivery catheter for one-way deployment.

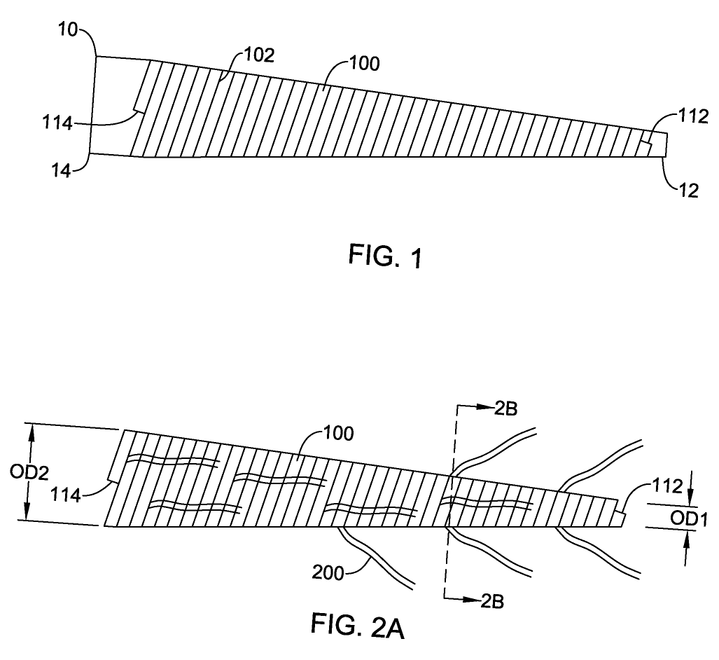

FIG. 1 shows an example embolic coil 100 made by winding a wire 102 around a tapered mandrel 10. The tapered mandrel 10 has a proximal end 12 with a first diameter and a distal end 14 with a second diameter larger than the first diameter. The embolic coil 100 may be formed from only a single continuous wire 102 with the coil having a proximal end 112 and a distal end 114. The wire 102 may be made of platinum and/or a platinum-containing alloy, including p latinum-Iridium, platinum- stainless steel alloys, etc . In other embodiments, the embolic coil 100 may be formed from multiple wires wrapped side by side or wrapped one over the other. In the embodiment shown in FIG. 1, the tapered mandrel 10 has a continuous gradual taper extending from the proximal end 12 to the distal end 14. Forming the embolic coil 100 on such a tapered mandrel 10 results in the embolic coil 100 having an outer diameter in a primary shape that increases uniformly from the proximal end 112 to the distal end 114. A uniform increase in diameter means the diameter increases continuously and gradually. In other embodiments, the mandrel may have one or more stepped changes in diameter, resulting in an embolic coil with a stepped change in outer diameter along its length.

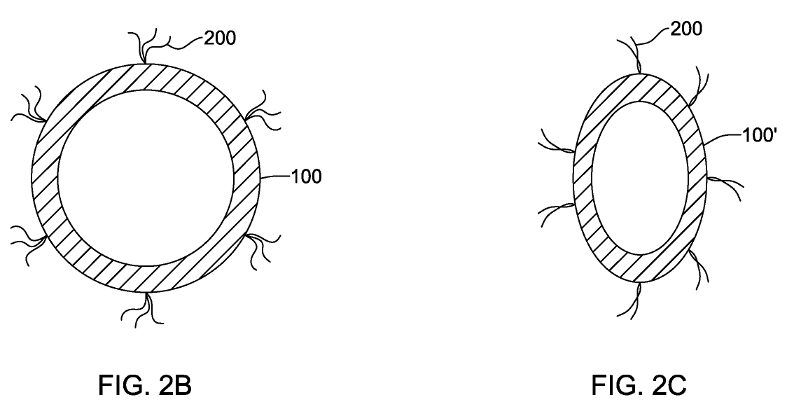

FIG. 2A illustrates a side view of the embolic coil of FIG. 1 released from the mandrel. Winding the continuous wire 102 on the tapered mandrel 10 provides a primary shape to the embolic coil 100 that is a spiral having an outer diameter that increases from a first outer diameter OD1 at the proximal end 112 to a second outer diameter OD2 at the distal end 114. OD2 is larger than OD1, which creates a tapered spiral as the primary shape, as shown in FIG. 2A. For an embolic coil 100 compatible with a delivery catheter having an inner diameter greater than 0.035 inch (0.889 mm), the first outer diameter OD1 may be between 0.02 inch (0.508 mm) and 0.025 inch (0.635 mm) and the second outer diameter OD2 may be between 0.023 inch (0.584 mm) and 0.03 inch (0.762 mm). For an embolic coil 100 compatible with a delivery catheter having an inner diameter between 0.021 inch (0.533 mm) and 0.027 inch (0.686 mm), the first outer diameter OD1 may be between 0.014 inch (0.355 mm) and 0.017 inch (0.432 mm) and the second outer diameter OD2 may be between 0.016 inch (0.406 mm) and 0.019 inch (0.483 mm). The overall length of the embolic coil 100 in its primary shape on the tapered mandrel 10 may be between 0.5 cm (0.2 inch) and 100 cm (39.37 inches).

Winding the wire 102 around the tapered mandrel 10 may create a gradient stiffness profile in the embolic coil 100 that transitions from a first stiffness at the proximal end 112 to a second stiffness at the distal end 114 that is less than the first stiffness. This stiffness gradient remains when the embolic coil 100 is removed from the mandrel. The embolic coil 100 may have only the primary shape created by wrapping around the mandrel 10, and the embolic coil 100 may conform to and pack within the vessel with only the primary shape. In other embodiments, the embolic coil 100 may be formed into a secondary shape by winding around a larger mandrel or by folding or otherwise forming into a more complex shape, as described below. In the embolic coil 100 having a distal OD2 between 0.023 inch (0.584 mm) and 0.03 inch (0.762 mm), described above, the proximal end 112 with the smaller OD1 may be greater than about 61 percent stiffer than the distal end 114. In the embolic coil 100 having a distal OD2 between 0.016 inch (0.406 mm) and 0.019 inch (0.483 mm), described above, the proximal end 112 with the smaller OD1 may be greater than about 66 percent stiffer than the distal end 114. The stiffer proximal end 112 has a larger radial force, which provides improved anchoring to the vessel wall.

The embolic coil 100 has a softer distal end 114 with a larger outer diameter as compared to the proximal end 112 that is stiffer with a smaller outer diameter. The embolic coil 100 is a multi-stiffness coil with the ability to anchor within tissue and form a shorter coil pack, thus shortening the final coil pack length. In situations where the embolic coil 100 is placed near a bifurcation, a shorter coil pack may prevent the proximal end from sticking out from the bifurcation into neighboring arteries.

The stiffer proximal end 112 can also provide pushability enabling the embolic coil 100 to navigate an often tortuous vascular path to a treatment site, while the more flexible distal end 114 may prevent injury as the embolic coil 100 is delivered out of the delivery catheter. Additionally, the stiffer proximal end 112 may be resistant to stretching if the embolic coil 100 needs to be retracted into the delivery catheter.

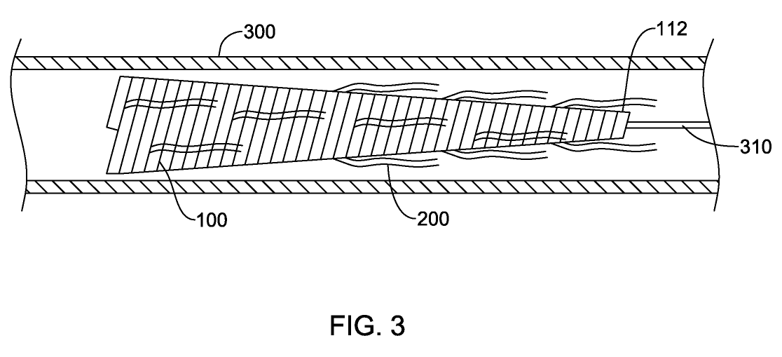

In the embodiment shown in FIG. 2A, the embolic coil includes a plurality of fibers 200 attached to the windings of the coil. The plurality of fibers 200 may be attached between windings of the embolic coil 100 by winding the middle part of each elongate fiber around the wire 102 such that the middle section of each fiber 200 is sandwiched between adjacent windings with both free ends of the fiber 200 extending freely, as shown in FIG. 2A. Multiple fibers 200 may be attached in a bundle at a single location, thereby increasing the surface area and thrombogenicity of the fibers. Each of the plurality of fibers 200 may be between 3 mm (0.12 inch) and 20 mm (0.78 inch) in total length, such that when attached to the embolic coil 100, the two free ends extend about 1.5 mm (0.06 inch) to 10 mm (0.4 inch) from the coil. The plurality of fibers 200 may be attached at random positions along the length of the embolic coil 100. In other embodiments, the plurality of fibers 200 may be attached to the embolic coil 100 in a uniform pattern. In some embodiments, the plurality of fibers 200 may be attached only to a proximal section of the embolic coil 100, as shown in FIG. 2A. The proximal section may be defined as any portion of the embolic coil 100 extending from the proximal end 112 towards and up to a midpoint between the proximal end 112 and the distal end 114. In other embodiments, the plurality of fibers 200 may be attached along the entirety of the length of the embolic coil 100, as shown in FIG. 3. The plurality of fibers 200 may be attached at random locations around the entire circumference of the embolic coil 100. The plurality of fibers 200 may be made of thrombogenic fibers which enhance thrombosis. In some embodiments, the plurality of fibers 200 may be formed from a polymer, including at least one of polyester, nylon, and polypropylene.

The tapered mandrel 10 may have a circular transverse cross-sectional shape, resulting in the embolic coil 100 having a circular cross-section, as shown in FIG. 2B which is a cross-sectional view taken through line 2B-2B in FIG. 2A. In other embodiments, the cross-sectional shape of the tapered mandrel and resulting embolic coil 100’ may be oval, as shown in FIG. 2C, or stadium shaped or any other generally rounded shape. When the embolic coil 100 is disposed within a delivery catheter 300, it will be constrained to have the primary shape, as shown in FIG. 3 which illustrates the embolic coil of FIG. 2A within a delivery catheter .

An embolization system may include the delivery catheter 300 and the embolic coil 100 releasably coupled to the delivery catheter. The embolic coil 100 is inserted into the delivery catheter 300 in the primary shape, and moves into the secondary shape upon exiting the delivery catheter. The embolic coil 100 may be delivered out of the delivery catheter 300 by a delivery wire 310, as shown in FIG. 3. In some embodiments, the proximal end 112 of the embolic coil 100 may include a first part of a coupling mechanism and the distal end of the delivery wire 310 may include a second part of the coupling mechanism (not shown), with the first and second parts of the coupling mechanism configured to have a coupled state and an uncoupled state. For example, the coupling mechanism may involve a friction fit, a threaded engagement, hooks, or any other structure that allows the proximal end 112 of the embolic coil 100 to be coupled to the delivery wire 310 during delivery, and to be released from the delivery wire 310 once the embolic coil 100 exits the delivery catheter 300. The releasable coupling of the delivery wire 310 to the embolic coil 100 may allow for the embolic coil 100 to be partially delivered out of the delivery catheter 300, withdrawn back into the delivery catheter 300 if the position needs to be adjusted, and then fully delivered out of the delivery catheter 300. In other embodiments, the proximal end 112 of the embolic coil 100 may not have any coupling structure, and instead the distal end of the delivery wire 310 may simply push the embolic coil 100 out of the delivery catheter 300. This may provide for a simpler delivery, but is a one-way delivery, with no mechanism for retrieving the embolic coil 100 once it exits the delivery catheter 300.

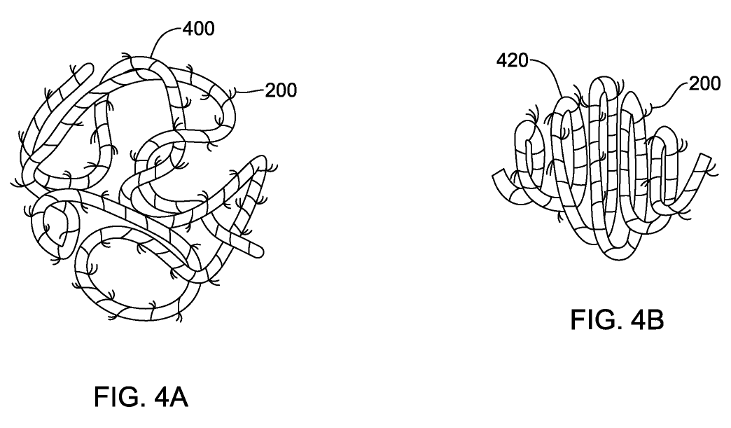

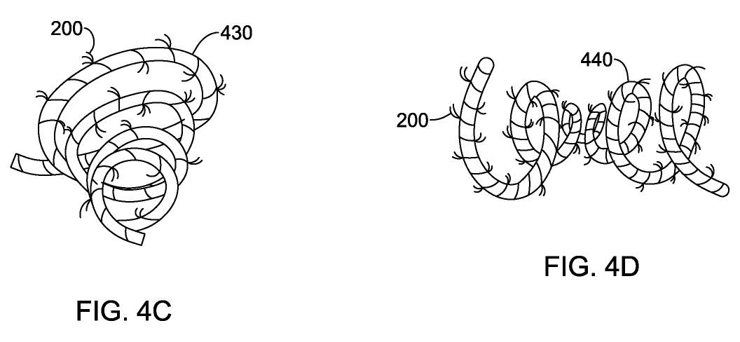

Once the embolic coil 100 exits the delivery catheter 300, the embolic coil 100 moves into at least one loop or fold in a secondary shape. This secondary shape may be achieved by wrapping the primary coil around a different mandrel or otherwise folding or looping the embolic coil 100 after removing the embolic coil 100 from the tapered mandrel 10. The secondary shape is generally a non-linear complex shape. For many procedures, particularly those involving occlusion of a flowing vascular stream, the secondary shape may help to assure effective embolization. The secondary shape of the embolic coil 100 may include at least one loop or fold formed from the spiral of the primary shape. Once the embolic coil 100 is formed into a secondary shape, it may be heat set to retain the secondary shape. FIGS. 4A-4D illustrate example secondary shapes for the embolic coil. FIG. 4A shows an embolic coil 400 having a basket-shaped secondary shape, FIG. 4B shows an embolic coil 420 having a diamond-shaped secondary shape, FIG. 4C shows an embolic coil 430 having a vortex or conical-shaped secondary shape, and FIG. 4D shows an embolic coil 440 with a dual-spiral secondary shape having two conical shapes that connect at their smaller ends.

A method of manufacturing the embolic coil 100 may include providing the tapered mandrel 10 with a tapered outer diameter and wrapping a continuous wire 102 around the mandrel to form a primary coil having a proximal end with a first outer diameter and a distal end having a second outer diameter larger than the first diameter. Wrapping the wire 102 around the tapered outer diameter of the mandrel creates a gradient stiffness profile in the primary coil when it is released from the mandrel. After the primary shaped embolic coil 100 is formed, a secondary shape may be formed that includes at least one loop or fold in the primary coil. The embolic coil 100 may then be heat treated to fix the secondary shape. In some embodiments, a plurality of fibers 200 may be attached to the embolic coil 100, either when in the primary shape, as shown in FIG. 2A, or after heat setting in the secondary shape, as shown in FIGS. 4A-4D. The embolic coil 100 may be devoid of any structure on its outermost surface other than the plurality of fibers 200.

A method of using the embolic coil 100 may include loading the embolic coil 100 into a delivery catheter 300 and inserting the delivery catheter 300 into a patient’s vasculature to a treatment site. At the treatment site, the embolic coil 100 may be pushed out of the delivery catheter using the delivery wire 310 which may then be uncoupled from the embolic coil 100, or the delivery catheter 300 may be withdrawn proximally leaving the embolic coil 100 in place at the treatment site, where the embolic coil 100 moves into the secondary shape. In some embodiments, a flowable material may be delivered with the embolic coil 100 such as, but not limited to OBSIDIO™ Conformable Embolic available from Boston Scientific of Marlborough, MA USA. The flowable material may be delivered after the embolic coil 100 has been released from the delivery catheter 300 and assumes its secondary shape.

It will be understood that the dimensions described in association with the above figure are illustrative only, and that other dimensions are contemplated. The materials that can be used for the various components of the embolic coil 100 and delivery system and the various elements thereof disclosed herein may include those commonly associated with medical devices. For simplicity purposes, the following discussion refers to the embolic coil 100 and delivery catheter 300 (and variations, systems or components disclosed herein). However, this is not intended to limit the devices and methods described herein, as the discussion may be applied to other elements, members, components, or devices disclosed herein.

In some embodiments, the embolic coil 100, delivery catheter 300, and delivery wire 310 (and variations, systems or components thereof disclosed herein) may be made from a metal, metal alloy, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, other nickel alloys such as nickel-chromium-molybdenum alloys (e.g., UNS: N06625 such as INCONEL® 625, UNS: N06022 such as HASTELLOY ® C-22® , UNS: N10276 such as HASTELLOY ® C276®, other HASTELLOY® alloys, and the like), nickel-copper alloys (e.g., UNS: N04400 such as MONEL® 400, NICKELVAC ® 400, NICORROS ® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

In at least some embodiments, portions or all of the embolic coil 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the embolic coil 100 (and variations, systems or components thereof disclosed herein). Some examples of radiopaque materials can include, but are not limited to, gold, platinum, palladium, tantalum, tungsten alloy, polymer material loaded with a radiopaque filler, and the like.

In some embodiments, the embolic coil 100, fibers 200, and/or delivery catheter 300 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene- b -isobutylene- b -styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An embolic coil comprising:
a coil having a proximal end and a distal end and formed from a continuous wire, the coil having a primary shape formed by winding the continuous wire on a tapered mandrel such that the primary shape of the coil is a spiral having an outer diameter that increases from a first outer diameter at the proximal end to a second outer diameter at the distal end that is larger than the first outer diameter, the coil having the primary shape when constrained within a delivery catheter;
wherein the coil has a gradient stiffness profile transitioning from a first stiffness at the proximal end to a second stiffness at the distal end that is less than the first stiffness; and
wherein the coil has a secondary shape after exiting the delivery catheter, the secondary shape including at least one loop or fold formed from the spiral of the primary shape.

2. The embolic coil of claim 1, wherein the outer diameter in the primary shape increases uniformly from the proximal end to the distal end.

3. The embolic coil of claim 1, further comprising a plurality of fibers attached between windings of the spiral in the primary shape.

4. The embolic coil of claim 3, wherein the plurality of fibers is attached only to a proximal section of the coil.

5. The embolic coil of claim 3, wherein the plurality of fibers is formed from at least one of polyester, nylon, and polypropylene.

6. The embolic coil of claim 1, wherein the continuous wire comprises a platinum-containing alloy.

7. The embolic coil of claim 1, wherein the first outer diameter is between 0.02 inch and 0.025 inch and the second outer diameter is between 0.023 inch and 0.03 inch.

8. The embolic coil of claim 1, wherein the tapered mandrel has an oval cross-section.

9. An embolization system comprising:
a delivery catheter;
an embolic coil releasably coupled to the delivery catheter, the embolic coil configured to be disposed within a lumen of the delivery catheter in a primary shape, and to form a secondary shape upon exiting the delivery catheter, the embolic coil having a proximal end and a distal end and formed from a continuous wire, the primary shape of the embolic coil formed by winding the continuous wire on a tapered mandrel such that the primary shape of the embolic coil is a spiral having an outer diameter that increases from a first outer diameter at the proximal end to a second outer diameter at the distal end that is larger than the first outer diameter; and
wherein the embolic coil has a gradient stiffness profile transitioning from a first stiffness at the proximal end to a second stiffness at the distal end that is less than the first stiffness.

10. The embolization system of claim 9, wherein the secondary shape is formed by releasing the embolic coil from the tapered mandrel and moving the embolic coil into at least one loop or fold and heat treating the secondary shape.

11. The embolization system of claim 9, wherein the outer diameter in the primary shape increases uniformly from the proximal end to the distal end.

12. The embolization system of claim 9, wherein the embolic coil includes a plurality of polymeric fibers attached between windings of the spiral in the primary shape.

13. The embolization system of claim 12, wherein the plurality of polymeric fibers is attached only to a proximal section of the embolic coil.

14. The embolization system of claim 9, wherein the continuous wire comprises a platinum-containing alloy.

15. The embolization system of claim 9, wherein the first outer diameter is between 0.014 inch and 0.017 inch and the second outer diameter is between 0.016 inch and 0.019 inch.

16. The embolization system of claim 9, wherein the tapered mandrel has an oval cross-section.

17. A method of manufacturing an embolic coil, comprising:
providing a mandrel having a tapered outer diameter;
wrapping a continuous wire around the mandrel to form a primary coil having a proximal end having a first outer diameter and a distal end having a second outer diameter larger than the first outer diameter, wherein the tapered outer diameter of the mandrel creates a gradient stiffness profile in the primary coil when released from the mandrel;
removing the primary coil from the mandrel and forming a secondary shape including at least one loop or fold in the primary coil;
heat treating the primary coil to set the secondary shape; and
attaching a plurality of polymeric fibers between windings of the primary coil.

18. The method of claim 17, wherein the tapered outer diameter of the mandrel increases uniformly from the proximal end to the distal end.

19. The method of claim 17, wherein the mandrel has an oval cross-sectional shape.

20. The method of claim 17, wherein the first outer diameter is between 0.02 inch and 0.025 inch and the second outer diameter is between 0.023 inch and 0.03 inch.
